(19) United States
(12) Patent Application Publication
(10) Pub. No.: US 2004/0230258 A1
(43) Pub. Date: Nov. 18, 2004

(54) Method and apparatus for treating pseudofolliculitis barbae

(75) Inventors: Gregory B. Altshuler, Wilmington, MA (US); Ilya Yaroslavsky, Wilmington, MA (US); Andrei V. Erofeev, N. Andover, MA (US); Michael H. Smotrich, Andover, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Palomar Medical Technologies, Inc., Burlington, MA

(21) Appl. No.: 10/783,987

(22) Filed: Feb. 19, 2004

Related U.S. Application Data
(60) Provisional application No. 60/448,762, filed on Feb. 19, 2003.

(51) Int. Cl. A61N 1/00

(52) U.S. Cl. 607/88

(57) ABSTRACT

Methods and apparatus for hair treatment are disclosed which comprise applying electromagnetic radiation (EMR) to a skin treatment area to deposit energy in one or more hairs so as to modify a shape and/or chemical structure of at least a portion of the hairs. The applied radiation can cause heating of the hair tips, so as to modify their shape, e.g., reduce sharpness of the hair tips. Modification of the hair can involve heat-induced changes to the shape, composition, or function of the hair tip, hair shaft, and/or hair matrix that make the hair less capable of re-entering the skin. The methods and apparatus can treat and/or prevent pseudofolliculitis barbae (PFB) in the treatment area. A method is also disclosed for managing hair growth using wavelengths between 1200 nm and 1400 nm.

### PRIORITY

This application claims priority to U.S. provisional application No. 60/448,762 filed Feb. 19, 2003.

### BACKGROUND OF THE INVENTION

The present invention is generally directed to hair treatment methods, and more particulary, to methods and apparatus for treatment and prevention of pseudofolliculitis barbae (PFB) by utilizing electromagnetic radiation.

Pseudofolliculitis barbae (PFB) is a chronic papulopustular dermatitis of a bearded area resulting from reentry penetration of the epidermis by a growing hair. PFB occurs more prevalently in persons (males and females) having curly hair. Persons of darker (IV to VI) skin types are also particularly susceptible to this condition. Epidemiological studies (P K Perry et al. J. Am. Acad. Dermatol., 46:S113-S119, 2002) give estimates of incidence between 45% and 83% for black patients.

Pathogenesis of PFB is determined by a person's hair structure. The curved pattern of the hair growth is the principal characteristic that initiates the process. In persons having such a pattern of hair growth, the hair emerges from the skin surface and turns in the direction of the epidermis. The growth continues in a direction as if to complete a full circle (i.e., extrafollicular penetration), resulting in the hair penetrating into the skin. A foreign-body-type inflammatory reaction that follows produces a plurality of papules and, in a continuing spectrum, pustules. Alternatively, the emerging hair penetrates the wall of the follicle rather than arcing across a portion of skin prior to reentry (i.e., transfollicular penetration).

Conventional treatment approaches include 1) beard growing; 2) PFB-specific shaving techniques; 3) application of depilatories and topical creams (e.g., U.S. Pat. No. 6,352,690); and 4) electrolysis for treatment of ingrown hairs (e.g., U.S. Pat. No. 5,419,344).

Recently, laser-based treatment modalities, initially developed for removal of unwanted hair, have been applied for treatment of PFB. The conventional treatment modalities, however, suffer from a number of short comings. In particular, beard growing is not an option for many occupations and PFB-specific shaving techniques are cumbersome, time-consuming, and often not sufficiently effective. Topical depilatories can be difficult to use and may cause severe skin irritation, exacerbating the condition. Electrolysis can only be performed by a trained professional, is expensive and extremely time-consuming. Laser modalities do offer a curative solution to the problem; however, they are currently only available at medical facilities, and existing systems may be sub-optimal for patients with darker skin types.

Thus, there exists an need in the art for a safe, effective, self-treatment method of PFB.

### SUMMARY OF THE INVENTION

In one aspect, the present invention provides a hair treatment method comprising applying electromagnetic radiation (EMR) to a skin treatment area to deposit energy in one or more hair tips in the area so as to modify at least a portion of the hair tips. The applied radiation can cause heating of the hair tips, which can extend, for example, from about 0.2 mm below the skin surface to about 1 mm above the skin surface, so as to modify their shape, e.g., reduce sharpness of the hair tips. Modification of the hair tip can involve heat-induced changes to the shape of the hair tip that make the hair less capable of re-entering the skin (i.e., causing a substantially rounded end of the treated hair tip). Thus, the applied radiation can treat and/or prevent pseudofolliculitis barbae (PFB) in the treatment area. More particularly, modification of the shape of the hair tips can inhibit extrafollicular and/or transfollicular penetration by the hair tips. In some embodiments, the applied radiation can cause irreversible thermal damage to any of the cortex and/or cuticle of the hair tips.

The applied radiation can raise the temperature of the hair tips to a range of about 50 to about 300° C. Parameters of the radiation may be selected so as to raise the temperature of the hair tips to a range of about 50 to about 300° C. while keeping epidermal temperature in the treatment area below about 65° C. and preferably below 60° C. or 55° C. A plurality of electromagnetic pulses can be directed to the treatment area so as to apply a fluence in a range of about 0.01 J/cm² to about 1000 J/cm² to the treatment area. The pulses can have pulse widths in a range of about 1 ns to about 5 minute or between about 1 ns to about 1 minute. The pulses can have a repetition rate of 0.1 Hz to about 10 MHz. Typically, the pulses are applied during a treatment session lasting for about 1 ns to about 100 seconds per cm² of the treated area. Preferably, the applied radiation includes wavelength components absorbed by melanin in the hair tips. For example, the radiation can include wavelength components in a range of about 280 nm to about 100,000 nm, and more preferably in a range of about 360 nm to about 600 nm.

In a related aspect, the hair treatment method can include cooling the epidermis in the treatment area, for example, to enhance selective heating of the hair tips relative to the epidermis. The cooling step can be performed at any of prior, during or after application of the radiation to the treatment area and may be used to prevent the epidermal temperature in the treatment from increasing to dangerous or uncomfortable levels, i.e., above 100° C.

The method of the present invention can further include applying a topical agent to the skin treatment area, where the topical agent can be photoactivated by the radiation to facilitate modifying the shape of the hair tips. The topical agent can include at least one exogenous chromophore, and optionally a vehicle for delivering the exogenous chromophore to the hairs, themselves, or to the pilosebaceous canal of hairs in the treatment area. The exogenous chromophore can be selected to have an absorption spectrum that at least partially matches the wavelength of the applied radiation so as to facilitate heating of the hair tips.

In further aspects, the hair treatment method can include depilating the treatment area. Depilation can be performed by shaving, clipping, applying a depilatory cream, applying additional electromagnetic radiation, or any other suitable technique. For example, the depilating step, which can remove portions of the hair tips protruding above the skin surface, can be performed by applying a plurality of electromagnetic pulses to the treatment area, either before or after applying the treatment pulses of electromagnetic radiation, or substantially simultaneously with applying the treatment pulses of electromagnetic radiation.

The hair treatment method can also include stretching the skin treatment area before or during treatment. The method may also include lifting the skin treatment area so that the hair tips are more accessible to the applied radiation. The hair tips themselves can be lifted so as to bring them into more direct contact with the applied radiation via any suitable mechanism, such as mechanical, vacuum, or electrostatic mechanisms.

In other aspects, the present invention also provides a method of treating hair by applying electromagnetic radiation to a skin treatment area for heating one or more hair shafts in the treatment area to a temperature sufficiently elevated so as to modify the hair shafts. The modification of the hair shafts can cause decreased curling of the hair shafts (i.e., substantial straightening of the hair shafts). The modification may also include increasing the softness of the hair shafts, changing the diameter or shape of the hair, increasing the tensile strength of the hair, and/or increasing the elasticity of the hair. The elevated temperature can be, for example, in a range of about 50° C. to about 300° C. The radiation can also cause a change in a tensile strength of the hair shafts. The change in the tensile strength can be in a range of about 1 to about 200 MPa of breaking stress. The radiation can provide sufficient modification of the hair shafts (i.e., reduction in the curling of the hair shafts) so as to treat, prevent or reduce pseudofolliculitis barbae (PFB) in the treatment area. The applied electromagnetic radiation can be delivered to the skin treatment area via a plurality of electromagnetic pulses having wavelength components in a range of about 380 nm to about 2700 nm, preferably about 600 to about 1400 nm, or about 800 to about 1350 nm. Further, the epidermis in the treatment area can be cooled prior to, during and/or after treatment. In addition, hairs in the treatment area can be substantially straightened prior to application of the electromagnetic radiation and/or a topical agent capable of photoactivation by the radiation can be applied to the treatment area to facilitate softening and/or straightening of the hair shafts.

In another aspect, the present invention provides a method of controlling hair growth by applying electromagnetic radiation having wavelength components in a range of about 1200 to about 1400 nm to one or more hair follicles in a skin treatment area so as to modulate hair growth. The applied radiation can cause a deceleration and/or cessation of hair growth. In some embodiments, the applied radiation can cause stimulation of hair growth. For example, the treatment area can be exposed to a plurality of electromagnetic pulses having pulse widths in a range of about 1 ns to about 1 minute to deliver radiation with a fluence in a range of about 0.1 J/cm² to about 1000 J/cm² to the treatment area. The duration and fluence of the applied radiation can be selected so as to cause heating of at least a portion of the hairs to a temperature greater than about 47° C. Further, the epidermis in the treatment area can be optionally cooled. A topical agent that is capable of photoactivation by the radiation can also be applied to the treatment area to facilitate modulating hair growth.

In yet another aspect, the present invention provides a method of treating hair that includes irradiating a plurality of hair follicles with radiation of a wavelength, and fluence suitable for causing the hair matrix to generate modified hair. The radiation can cause the hair matrix to effect growth of less curly, thinner and/or softer hair by heating the hair bulb, keratogenous zone or bulbar of the hair follicles. The modified hair can exhibit a change in a tensile strength in a range of about 1 to about 200 MPa of breaking stress relative to that of a pre-treatment hair. The thinner hair can exhibit a reduction in diameter in a range of about 1 to about 60 μm relative to that of a pre-treatment hair. The radiation can be delivered to the treatment area via a plurality of electromagnetic pulses having wavelength components in a range of about 380 nm to about 2700 nm, more preferably about 600 to about 1400 nm and having pulse widths, e.g., in a range of about 1 ns to about 1 minute, so as to expose the treatment area to a fluence in a range of about 0.1 J/cm² to about 1000 J/cm², or more preferably a fluence in a range of about 5 to about 50 J/cm².

In another aspect, the invention provides an apparatus for treating a skin treatment area that includes a radiation source for applying one or more pulses of electromagnetic radiation (EMR) to the skin treatment area to deposit energy in one or more hair tips so as to modify (i.e., change the shape, alter the tensile strength or texture, soften, straighten) at least a portion of the hair tips and a depilating mechanism for depilating at least a portion of the skin treatment area. The depilating mechanism can include implements for shaving, applying a depilatory cream, applying additional electromagnetic radiation, or any hair removing mechanism known in the art. The radiation source can generate electromagnetic pulses having wavelength components in a range of about 300 nm to about 1900 nm. The apparatus can also include a cooling mechanism for cooling epidermis in the treatment area before, during and/or after treatment. The apparatus can also have a sensor for sensing removal of the hair tips protruding above the skin surface and/or a lifting mechanism for enhancing capture of the hair tips by the cutting mechanism. The lifting mechanism can be mechanical and/or electrostatic.

In another aspect, the present invention also provides an apparatus for controlling hair growth, comprising a radiation source for applying electromagnetic radiation having wavelength components in a range of about 1200 to about 1400 nm to one or more hair follicles in a skin treatment area so as to modulate hair growth.

In another embodiment, the invention provides an apparatus for modifying a shape of a least a portion of a hair tip, comprising at least a radiation source generating radiation pulses having wavelengths in a range of about 280 nm to about 100,000 nm and pulse widths in a range of about 1 nsec to about 5 minutes to illuminate a skin treatment area with a fluence in a range of about 0.01 J/cm² to about 1000 J/cm² so as to modify shapes of at least some hair tips in the treatment area. The invention also provides an apparatus for reducing curliness of hair shafts, comprising one or more radiation sources generating radiation pulses having wavelengths in a range of about 380 nm to about 2700 nm and pulse widths in a range of about 1 nsec to about 1 minute for illuminating a skin treatment area with a fluence in a range of about 0.1 J/cm² to about 1000 J/cm² so as to reduce curliness of at least some hair shafts in the treatment area. In another embodiment, the invention provides an apparatus for controlling hair growth, comprising at least one radiation source generating electromagnetic radiation having wavelength components in a range of about 1200 to about 1400 nm for application to one or more hair follicles in a skin treatment area so as to modulate hair growth, wherein the radiation source can be any of an LED, a laser diode, a filtered arc lamp or a filtered halogen lamp. The apparatuses described in this invention can also include a mechanism for removing portions of the hair tips protruding above the skin surface. In addition, the apparatus can include a positioning mechanism for positioning the hair for treatment. The positioning mechanism can be a mechanical, electrostatic, and/or vacuum source capable of moving a portion of the hair so that the hair can optimally receive the applied radiation.

In another embodiment, the invention provides an apparatus for modifying elasticity of hair shafts comprising one or more radiation sources generating radiation pulses having wavelengths in a range of about 600 to about 1400 nm and pulse widths in a range of about 1 nsec to about 1 minute for illuminating a skin treatment area with a fluence in a range of about 0.1 J/cm² to about 1000 J/cm² so as to modify elasticity of at least some hair shafts in the treatment area.

In yet another embodiment, the invention provides a dermatological system comprising an applicator having a head portion adapted for scanning over a skin treatment area and incorporating at least one radiation source, a tracker coupled to the head portion for generating signals indicative of positions of the head portion during a scan, and a controller coupled to the tracker and the radiation source, the controller periodically activating the radiation source based on position signals received from the tracker. The controller determines a distance traversed by the head portion since a previous activation of the radiation source based on the position signals. The controller activates the source when the traversed distance exceeds a threshold.

### BRIEF DESCRIPTION OF THE DRAWINGS

FIG. 1A is a picture of a hair tip before EMR treatment;

FIG. 1B is a picture of the hair tip after EMR treatment;

FIG. 2 is a graph illustrating the results of EMR treatment for skin type VI;

FIG. 3A is a photograph of a section of a person's leg before treatment;

FIG. 3B is a photograph of the section of a person's leg 3 months after treatment showing changes in the hair shafts;

FIG. 4 is an absorption spectrum of melanin between 1000 nm and 1400 nm;

FIG. 5 is a graph comparing the change in temperature of the hair tip and the basal layer of skin following irradiation at various wavelengths;

FIG. 6A is a graph of the transmittance of skin from the skin surface to the hair bulb as as a function of wavelength for skin with the waveguide effect in a light hair (1); skin with the waveguide effect in a dark hair (2); and skin without the waveguide effect (3).

FIG. 6B is a graph of the ratio of the temperature raise at the hair matrix to that of the basal layer of the epidermis (“safety ratio”) accounting for the waveguide effect as a function of wavelength for light hair (1), and for dark hair (2).

FIG. 7A is a schematic illustration of a “stamping” mode of delivering electromagnetic radiation to a skin treatment area;

FIG. 7B is a schematic illustration of a “scanning” mode of delivering electromagnetic radiation to a skin treatment area;

FIG. 7C is schematic illustration of a “matrix” mode of delivering electromagnetic radiation to a skin treatment area;

FIG. 8 is a schematic illustration of one embodiment of the present invention which utilizes a pulsed source of EMR in the scanning mode;

FIG. 9 is a schematic illustration of an embodiment of the present invention in which firing of a new EMR pulse is initiated based on a predefined triggering condition;

FIG. 10 is a schematic illustration of an embodiment of the present invention in which a plurality of EMR sources are organized in a linear array in a handpiece;

FIG. 11 is a schematic illustration of an embodiment of the present invention in which the EMR sources are positioned in a rotating drum;

FIG. 12 is a schematic illustration of an embodiment of the present invention in which additional implements are included in the apparatus;

FIG. 13 is a schematic illustration of the experimental set-up used in Example 1;

FIG. 14A is a graph of the temperature profile of hairs in air following EMR treatment at 1060 nm;

FIG. 14A is a graph of the temperature profile of hairs in air following EMR treatment at 1208 nm; and

FIG. 15 is a graph of the ratio of melanin to water absorption as a function of wavelength.

### DETAILED DESCRIPTION OF THE INVENTION

The present invention discloses methods of modifying the hair shaft that directly address the cause of PFB, i.e. the hair curliness and the sharp end of the hair resulting from hair plucking and/or shaving. These potential hair shaft modification solutions can advantageously result in a simple inexpensive PFB cure. As disclosed by the present invention, hair shaft modification can be achieved through at least one of the following methods: 1) thermo-induced changes in the structure of the shaft to modify the tensile properties of the hair in such a way that its tendency to curl decreases, 2) thermo-induced shrinkage of the hair shaft to reduce traction between the companion layer and the outer root sheath (ORS), which facilitates shedding of the hair, 3) thermo-induced changes in the companion layer reduce traction between the companion layer and the ORS that, in turn, facilitates shedding of the hair, and 4) thermo-induced changes in the shape (i.e., decreased sharpness) of the hair to decrease the probability of extrafollicular and/or transfollicular penetration.

According to aspects of the present invention, PFB can be treated or prevented by applying electromagnetic radiation (EMR) to a plurality of hair follicles or parts thereof in a skin treatment area. A method of treatment of PFB according to the present invention can include a step of examining and identifying portions of skin afflicted with PFB and selectively applying EMR to those regions.

PFB is a skin problem exaggerated by the imposition of environmental and appearance constraints placed on individuals having genetically imposed hair or hair follicles features which on shaving, provide the causal factors of PFB. Furthermore, PFB is not a true folliculitis, in that a pathogenic microorganism is not involved in its etiology. Rather, the basis of its etiology is a foreign-body-type inflammatory response. After close shaving, the sharp edge of the hair shaft transects the wall of the hair follicle or re-enters the epidermis. The present invention describes methods, and apparatus for implementing these methods, that can reduce, prevent and/or treat PFB in a subject.

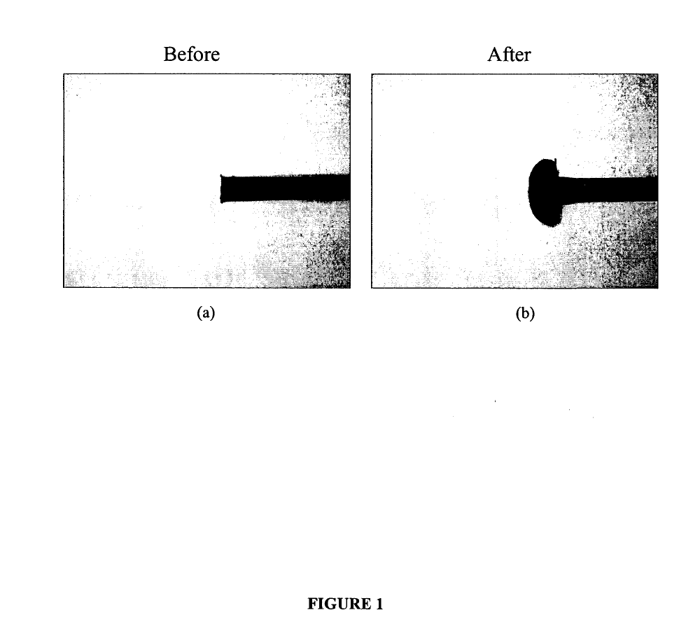

In one aspect of the invention, electromagnetic radiation (EMR) is applied to a plurality of hair tips such that the shapes of the hair tips are modulated. The term “hair tip” is known in the art, and as used herein generally refers to a portion of the hair that extends from below the skin surface in proximity of the surface to above the skin surface. For example, a hair tip can refer to the portion of the hair shaft extending from a depth of about 0.2 mm below the skin surface to about 1.0 mm above the skin surface. The hair tips are selectively heated to cause temporary or irreversible thermal damage or modification to the cortex and/or cuticle of a hair tip, such that the tip assumes a modified shape. Modification of the hair tip involves heat-induced changes to the shape of the hair tip that make the hair less capable of re-entering. More particularly, the modified shape can be preferably less sharp, e.g., more rounded, than the unmodified hair tip. By of way of example, FIG. 1A and FIG. 1B provide a comparison, respectively, of a hair tip before a treatment according to the teachings of the invention with a hair tip having a more rounded tip caused by exposure to electromagnetic radiation in accordance with the teachings of the invention, as described in more detail below. The modulation of the hair tip shape can be performed following or simultaneously with depilating the skin treatment area.

In one embodiment, EMR is projected onto the hair tip such that the hair tip reaches a temperature in a range of about 50° C. to about 300° C. In some embodiments, it is preferable that the temperature of the hair tip exceeds about 100° C. In other embodiments, it is preferable that the temperature of the hair tip exceeds about 200° C.

A modification to a hair tip according to this aspect of the invention can be achieved by utilizing EMR having a wavelength longer than about 280 nm. Preferably, the wavelength is in a range of about 280 to about 100,000 nm, more preferably in a range of about 280 to about 1400, and most preferably in the range of about 380 to about 600 nm. Wavelengths absorbed by melanin and/or water in the hair can be targeted for heating.

Heating of hair tips can be achieved selectively so that the underlying skin remains undamaged. This selectivity results from the differences in the heat dissipation characteristics of the hair tips and the skin. While the epidermis also includes areas of melanin, it is mostly in the basal membrane which is located deeper in the skin and has higher thermal contact with surrounded tissue, thereby the EMR is attenuated by the upper layers of the epidermis before reaching the basal membrane and providing substantial heat dissipation relative to the hair tip.

Further, heat from skin tissue (epidermis) can be removed much more efficiently than heat from a hair tip, due to high thermal conductivity of the surrounding tissues. The heat dissipation limits the temperature of the epidermis to below that of the hair tip. In some embodiments, the skin surface may be cleaned before treatment, to remove any thermal conductive material from the tip and/or surrounding areas, thereby enhancing the selective heating of the tip versus skin. In some embodiments, skin surface can be cooled to further ensure heat dissipation from the epidermis. For example, cooled or room-temperature air can be used as a cooling agent. In another aspect, the air flow is used to dry the hair and, thus, decrease the heat flux from the hair tip. In other embodiments, room-temperature or heated air can be supplied to the treatment area before, during, and/or after treatment.

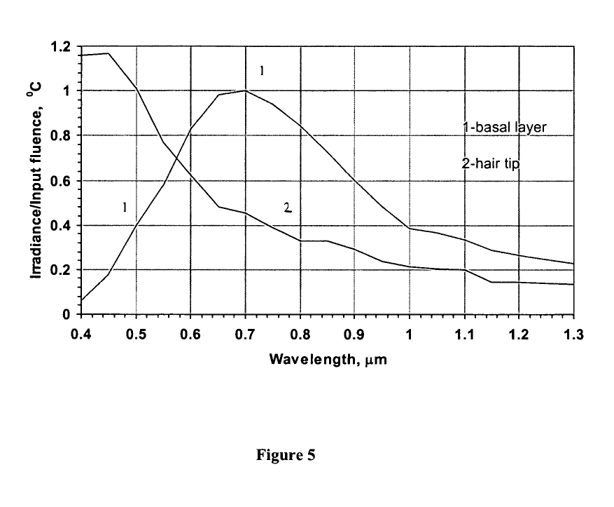

FIG. 5 is a graph comparing the change in temperature of the hair tip and epidermis following irradiation at various wavelengths. The most effective wavelengths for selective heating of hair tips are in UV and violet spectrum. Typical parameters for this treatment include wavelengths in the range of about 280 to about 100,000 nm, preferably in a range 360-600 nm to limit the penetration of the light into the basal layer of skin, a fluence in a range of about 0.01 J/cm² to 1000 J/cm², and more preferably in a range of about 0.5 to about 50 J/cm². In some embodiments, the electromagnetic energy is applied to the treatment area by exposure of the area to a plurality of electromagnetic pluses having a suitable wavelength, and pulse widths that are preferably shorter than the thermal relaxation time of the hair tip. Thermal relaxation time of the hair tip, which can depend on its diameter and dryness of surrounding medium can be in a range of, e.g., 1 ms to 10 s. Typically, shorter pulse widths are preferable so that his condition is better fulfilled. The particular pulse width, fluence and wavelength selected for a particular application depends on a number of characteristics, including, but not limited to, skin type and hair color. In some embodiments, the pulse width, fluence and wavelength selected for a given patient will typically deliver less EMR than would be necessary to achieve hair growth reduction or hair removal. Generally, pulse widths in a range of about 1 ns to about 5 minutes are employed.

Various sources of EMR can be used to practice the present invention. Examples include, but are not limited to diode lasers, including quantum-cascade lasers, solid-state lasers, LEDs or other solid-state lightings, an array or matrix of LEDS, arc lamps, halogen lamps, fiber lasers, metal halide lamps, incandescent lamps, RF generators, and microwave generators. The EMR source can produce pulsed or continuous radiation. In general, application of the EMR may be achieved using any suitable apparatus for delivering EMR according to the parameters described above. For example, the device may be structured similarly to a device as described in U.S. Pat. No. 6,517,532, U.S. Pat. No. 6,508,813, U.S. patent application Ser. No.: 10/154,756, entitled: “Cooling system for a Photocosmetic Device,” filed on May 23, 2002, U.S. patent application Ser. No. 10/702104, filed Nov. 4, 2003 entitled “Methods and Apparatus for Delivering Low Powered Optical Treatments,” U.S. patent application Ser. No. 10/080,652, filed Feb. 22, 2002, entitled “Apparatus and Method for Photocosmetic and Photodermatological Treatment,” U.S. patent application Ser. No. 10/706,721, filed Nov. 12, 2003 entitled “Method and Apparatus for Performing Optical Dermatology,” and U.S. Pat. No.: 6,514,242 entitled “Method and Apparatus for Laser Removal of Hair.”

In some embodiments, EMR is applied perpendicularly to the skin surface or at various angles. For example, it may be appropriate to apply the light obliquely or at a grazing angle, thus facilitating coupling of EMR into hairs growing at an oblique angle to the skin surface.

In a preferred embodiment, EMR will be applied after depilating the skin treatment area. The EMR can be applied after each shave or applied as needed (i.e., following every other shave). Depilating may be achieved by using any suitable mechanism for removing at least a portion of the hair tips protruding above the skin. Examples of suitable depilating mechanisms include, but are not limited to, shaving, clipping, applying a depilatory cream, and applying additional electromagnetic radiation. In an preferred embodiment, depilation is achieved through shaving using any suitable apparatus (e.g., a blade or electric razor). Typically, at the time of EMR application, the tip will be located at a depth in a range of 0.2 mm below the skin surface, to 1.0 mm above the surface.

In some embodiments, the skin may be stretched prior to treatment such that the hair tips are more accessible to the applied EMR. In other embodiments, a means for mechanical or electrostatic capture of the hair tips can be used in order to bring them into an optimal position for treatment. Alternatively, the EMR can act also as cutting tool, combining clipping and tip processing in a single pass. Air flow, which may be heated, cooled, or at room temperature, can be delivered to the skin treatment area to dry hair tips before EMR exposure.

In some embodiments, EMR can be applied at the same time or directly prior to using a straightening implement to align the hair shaft into a straight position after it has been heated and softened by EMR. Such an implement can utilize, for example, mechanical, electrostatic, or chemical action (or combination thereof). Alternatively, a topical substance capable of straightening hair can be applied to the skin treatment area before, during or after EMR treatment. Various hair straighteners are known in the art (See for example, U.S. Pat. Nos. 6,537,564 and 6,517,822). Most available hair straighteners are either hydroxide based, with, for example, sodium hydroxide, calcium hydroxide and potassium hydroxide as the active ingredient, or ammonium thioglycolate based.

In some embodiments, topically applied chromophores are employed to facilitate treatment by electromagnetic radiation. The wavelength of EMR can be optimized in order to at least partially match the absorption spectrum of the chromophore. Treatment can also be enhanced by applying the chromophore using a delivery system that provides penetration of the chromophores into the pilosebaceous canal and/or hair shaft. The chromophore may be an organic or non-organic dye in combination with a vehicle. In some embodiments, a topically applied depilatory agent may be applied to facilitate treatment. The depilatory agent may be light or heat activated, such that by concentrating (e.g., focusing) the EMR at the depth of the hair tips, the depilatory agent can be selectively activated in the region of the hair tip. Optionally, the topical composition may contain both the depilatory agent and a chromophore agent for the EMR. The chromophore agent can be selected from the group consisting of dyes, metals, ions, colored particles, photosensitive dyes, photosensitive materials, carbon particles, conductive skin lotions, electrolyte sprays, conductive electrode gels, and oxides. For examples of topical substances, see for example, U.S. Pat. No. 6,685,927, U.S. patent application Ser. No. 10/693682, filed Oct. 23, 2003 entitled “Phototreatment Device for Use with Coolants and Topical Substances,” which is hereby incorporated by reference in its entirety.

In another aspect of the invention, the hair shaft becomes less curly. “Curly” or “curliness” as used herein refers to a combination of the ability of the hair to form a curved line (loop) and the lack of elasticity of the hair shaft. A decrease in the curliness of the hair can also lead to an increase in the softness of the hair, preferably in the infundibulum area, a modification of the diameter or shape of the hair, an increase in the tensile strength of the hair, and/or an increase in the elasticity of the hair. Thus, the physical and chemical nature of the hair shaft is modified through the application of EMR which heats the hair tips to a temperature in the range of about 50° C. to about 300° C., preferably greater than 100° C., and more preferably greater than 200° C. Typical parameters for this treatment include wavelengths in the range of about 380 to about 2700 nm, preferably in a range of about 600-1400 nm, and more preferably in the range of about 800-1350 nm. As a result of the heating, the structure of the tip may change as the material of the tip becomes softer. The terms “soft” or “soften” as used herein are intended to refer to the thermal induced modification of the structure of the cuticle, cortex or intercellular cement of the hair shaft which decreases the hardness of the edge of the hair tips. Softness can be determined, for example, by measuring the tensile strength of the hair shaft. In some embodiments of the invention, the applied radiation can cause a change in the tensile strength of the hair shafts in a range of about 1 to about 200 MPa of breaking stress, and more preferably in a range of about 5 to about 100 MPa of breaking stress. In some embodiments, the applied radiation may provide not only a change in the physical and chemical properties of the hair shaft resulting in a change in the texture of the hair, but the shape of the hair tip may also be modified as described above.

The hair tips can be selectively heated to cause temporary or irreversible thermal damage or modification to the cortex and/or cuticle of a hair tip. As a result of this treatment, the hair cuticle and/or cortex and/or intercellular cement are modified (i.e., damaged), which can produce less curly hair, softer hair, thinner hair, an increase in the tensile strength of the hair, and/or an increase in the elasticity of the hair. Modification to the cortex and/or cuticle of a hair tip, according to this aspect of the invention, can be achieved using EMR with wavelengths longer than 380 nm. Preferably the wavelength is in a range of about 380 to about 2700 nm, and more preferably in a range of about 600 to about 1400 nm. The wavelength of light may be selected to selectively target lipids, water, melanin, and/or keratin (i.e., components of the hair shaft). Pre-cooling of the epidermis and cooling of the epidermis simultaneously with application of the EMR (known as “parallel” cooling) may also be employed to improve depth selectivity of such treatment. Cooling is more effective on high thermo conductive tissue, such as dermis and epidermis which are significantly more thermoconductive than the hair shaft due to their higher water content. Surface skin cooling is therefore more effective on the dermis than the hair shaft leading to selectivity for hair shaft heating. Selectivity of heating of hair shaft, which has low thermal conductivity, can be achieved by employing wavelengths in the range of about 380 to about 2700 nm.

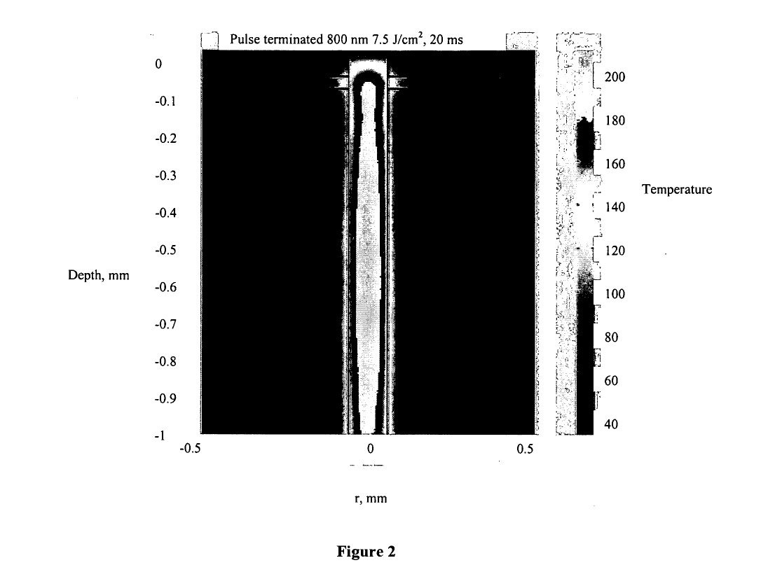

Typical parameters for this treatment include: a wavelength in the range of about 380 to about 2700 nm, a pulse width of about 1 ns to about 1 minute, and a fluence of about 0.1 to about 1000 J/cm². The pulse width, fluence and wavelength selected for a given patient will typically deliver less EMR than would be necessary to achieve hair growth removal or hair reduction. FIG. 2 shows exemplary results of this mode of treatment for skin type VI by employing a plurality of radiation pulses having a wavelength of 800 nm, a pulse width of 20 ms, and a fluence of 7.5 J/cm². As a result of this treatment, hair shaft at a depth of about 0 to 0.8 mm has been heated up to 200° C., while the epidermal temperature does not exceed 65° C. In some embodiments, the beam width may be selected to be relatively narrow to limit penetration to the depth of the hair shaft. The beam may be shaped as a circle, a line or any other suitable shape so as to limit the penetration using scattering. Some embodiments may utilize focusing the beam so that EMR is concentrated at a desired depth.

In yet another aspect of the invention, a method is provided to modify the hair bulb, keratogenous zone and/or bulbar of a hair follicle, via heating or cooling, to cause a change in the new hair growth. As a result of such heating or cooling, functions of the hair matrix can be affected. In particular, the hair growth process can be modified so as to lead to changes in the nature of the re-growing hair. For example, the newly grown hairs become softer and/or change their shape (reduce cross-section, i.e., become thinner; or increase ellipticity, i.e., become more round), which makes them less susceptible to curling. Also, the chemical structure of the newly grown hair can be modified to make the hair shafts substantially straighter. For selective heating of hair bulb and bulbar, EMR can be applied with wavelengths in the range of about 380 to about 2700, or more preferably in the range of about 600 nm to about 1400 nm, with pulse widths of about 1 ns to about 1 minute, and fluences of about 0.1 J/cm² to about 1000 J/cm², and more preferably in a range of about 1 to about 100 J/cm².

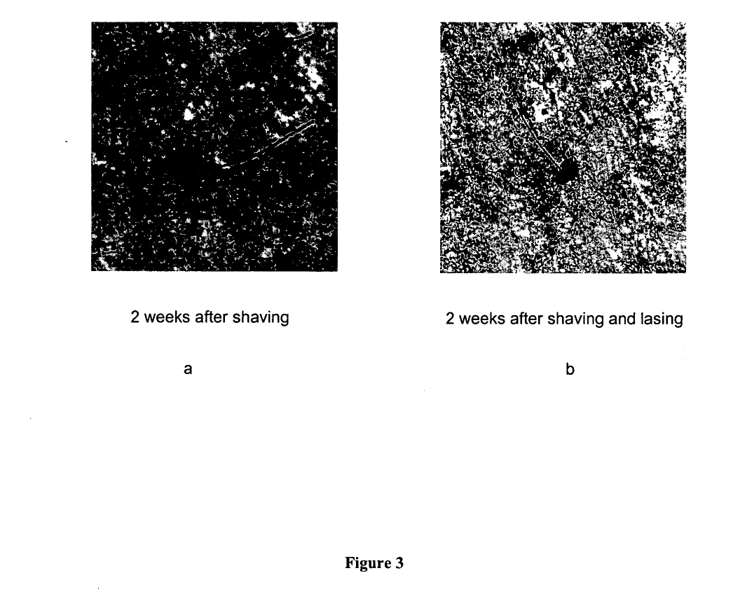

FIGS. 3A and 3B demonstrate the use of the present invention for altering the chemical and physical properties of the hair shafts. An example of hair miniaturization following treatment with EMR is shown through the differences in FIG. 3A (before treatment) and FIG. 3B (after treatment). EMR treatment was applied using a broadband source (wavelengths between 530 and 1200 nm), a fluence of around 12 J/cm² and a pulse width of 20 ms. The pulse width, fluence and wavelength used for a given patient will typically deliver less EMR than would be necessary to achieve hair growth removal or hair reduction for that patient. Selective absorption and/or conductivity and/or thermal property of the bulbar versus surrounded tissue enables selective heating of the bulbar.

In some embodiments, the subcutaneous area can be cooled at the depth of hair bulbar location to a temperature of about 5° C. to about 30° C. so that the hair matrix and/or dermal papilla or/and vascular loop will be selectively affected by EMR treatment. Cooling can be achieved through a variety of mechanisms known in the art such as spraying a cooling substance (i.e., cooled air or liquid), using a phase-change material, or contacting the target area with a cooling element. For example, contact cooling (i.e., by bringing a cooling element in contact with skin surface) can be employed. Alternatively, topical substances can be applied to the skin surface to selectively cool a portion of the treatment region (see, for example, U.S. patent application Ser. No.: 10/154,756, entitled: “Cooling system for a Photocosmetic Device,” filed on May 23, 2002 and U.S. patent application Ser. No. 10/693682, filed Oct. 23, 2003 entitled “Phototreatment Device for Use with Coolants and Topical Substances.”)

Further, similar to the above aspects of the invention, a topical agent, e.g., a lotion, can be applied to the skin treatment area to facilitate heating of hair bulb, keratogenous zone and/or bulbar of a hair follicle. The topical agent can include an exogenous chromophore that can penetrate into at least a portion of the hair follicle. The exogenous chromophore can preferably exhibit an absorption spectrum that at least partially matches the wavelength of the applied radiation so as to facilitate heating of the hair shaft.

In yet another aspect, the present invention provides methods for modifying hair growth of a subject. While, the use of EMR for hair removal (see, for example, U.S. Pat. No. 5,595,568) or for reduction of hair growth rate (see for example, WO Patent Application 2003/077783) is known in the art, the present invention discloses the use of a new range of wavelengths of this purpose. Previous methods and apparatus used optical radiation with wavelength shorter than 1200 nm. However, for darker skin types, treatment with wavelengths shorter than 1200 nm can result in unwanted side effects, such as epidermal damage. To overcome these drawbacks, the present invention recognizes that the wavelength range between about 1200 nm and about 1400 nm can be used to modify hair growth. The pulse width, fluence and/or power can be adjusted so that wavelengths in the range between about 1200 nm and about 1400 nm can be used to slow and/or reduce hair growth, stop hair growth or stimulate hair growth.

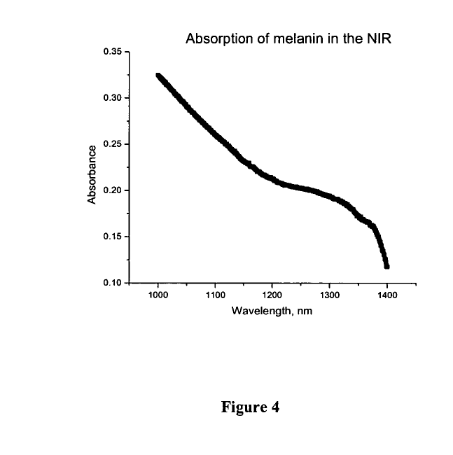

FIG. 4 is an absorption spectrum of melanin between 1000 nm and 1400 nm showing that melanin does absorb light in the near infrared spectrum. Feasibility of using wavelengths longer than 1200 nm for heating melanin-containing targets has been demonstrated in Example 1. Penetration of the optical radiation in the 1200-1400 nm wavelength range to the matrix of the follicle can be facilitated by a waveguide effect in the hair follicle structure. The waveguide effect is caused by a difference in the refractive index between the hair shaft, inner root sheath, out root sheath and surrounding tissue. Specifically, the refractive index of the shaft, inner root sheath, outer root sheath is substantially higher than that of tissue. As a result, light, with a wavelength in a range of about 1200 to 1400 nm coupled to the hair follicle through the infulbidum can propagate down the follicle in a series of total internal reflections (TIRs), which effectively increase the depth of penetration. This effect can be significant in areas of dense hair follicles, such as facial tissue where hair follicle density can be as high as 1000 hairs per cm². In areas of high hair follicle density, i.e., where more than 30% of the skin volume is occupied by hair follicles, use of a large beam results in a waveguide effect that helps propagate light through the bundle of hair follicle waveguides which can diffuse propagation through dermis. For coherent laser beam this structure can play a role similar to a photonics crystal with amplification of waveguide effect on certain wavelengths.

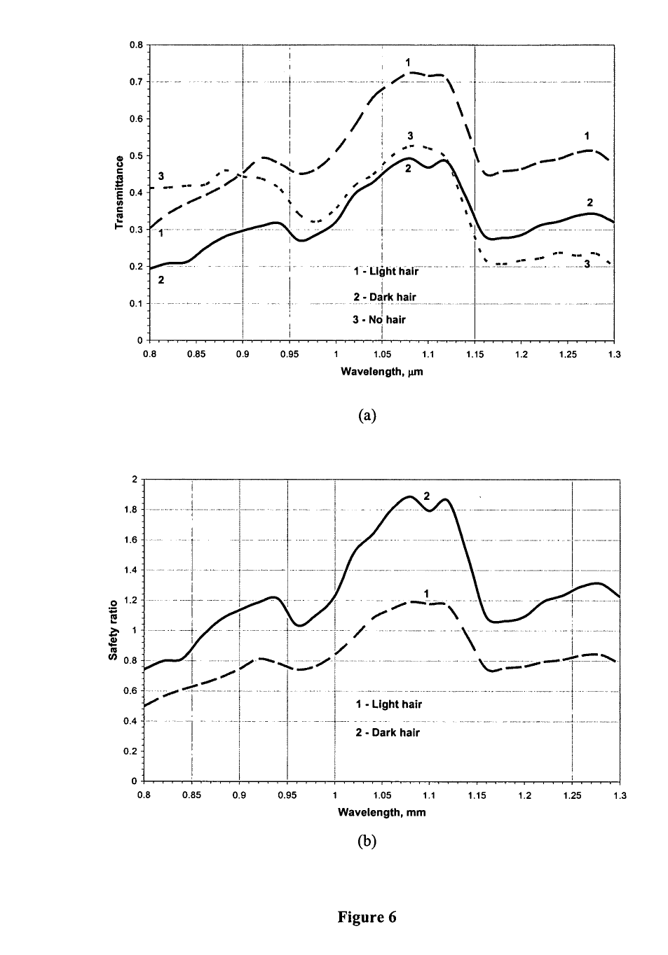

FIGS. 6A and 6B illustrate the impact of this waveguide effect on the amount of optical energy transmitted to the hair bulb. FIG. 6A is a graph of the transmittance of EMR in the skin from the surface to the location of the hair bulb (of a single follicle) as a function of wavelength for two cases: accounting for the waveguide effect (1) and neglecting the waveguide effect (2).

FIG. 6B is a graph of the ratio of the transmittance of skin with the waveguide effect to that of the skin without the waveguide effect as a function of wavelength. FIG. 6B shows that the waveguide effect is most pronounced and particularly advantageous as the wavelength of the applied radiation increases, e.g., longer than 1200 nm. Thus, substantial retardation of hair growth with the wavelengths between 1200 nm and 1400 nm can be achieved using fluences between about 1 J/cm² and 500 J/cm² and pulse widths between about 1 ns and 10 min.

The invention also provides apparatus for depilating and applying EMR to a skin treatment area as discussed below. In some embodiments, the depilating apparatus and the EMR delivery apparatus are located on a single device, such that in a single stroke the EMR is applied subsequent to or substantially simultaneous with depilation. The depilating apparatus may comprise any suitable device for hair removal known in the art, such as a mechanisms for shaving (i.e., a blade or an electrical razor), a mechanism for tweezing hairs (i.e., a rolling device that tweezes hairs as it passes over the skin treatment area), an applicator of depilatory cream, electrolysis, or an applicator of additional electromagnetic radiation. In a preferred embodiment, the EMR can act also as cutting tool, combining clipping and tip processing in a single pass. The apparatus can also contain mechanisms for mechanical or electrostatic capture of the hair tips in order to bring them into an optimal position for treatment. In yet other embodiments of the present invention, the apparatus can contain an air flow mechanism to deliver air to dry the hair tips before EMR exposure and/or a stretching mechanism to stretch the skin treatment area prior to EMR treatment.

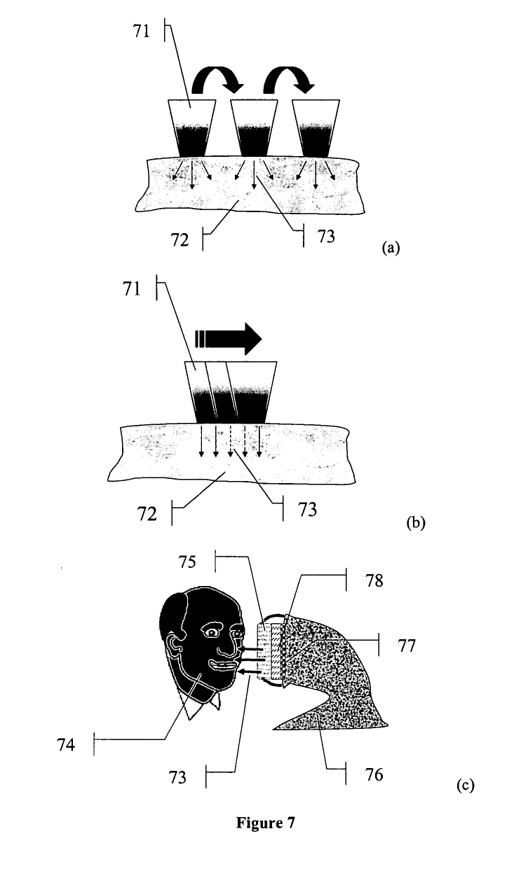

A variety of different designs can be adopted for an implementing apparatus for practicing the methods of the invention as described above. In particular, electromagnetic radiation can be applied to a skin treatment area in many different ways in various embodiments for practicing the methods of the invention. By way of example, FIGS. 7A, 7B, and 7C schematically present three exemplary modes of delivering electromagnetic radiation to a skin treatment area. With reference to FIG. 7A, in a “stamping” mode, an applicator (handpiece) 71 incorporating one or more sources of electromagnetic radiation can be placed on a selected area of skin 72, and a pulse of electromagnetic radiation 73 can be applied to the tissue in this area. The handpiece can then be moved to another portion of the treatment area to apply an EMR pulse to that portion. This process can be repeated until electromagnetic pulses are applied to the entire treatment area.

FIG. 7B schematically illustrates another mode of applying electromagnetic radiation to a treatment area, herein referred to as “scanning” mode, in which a handpiece incorporating one or more electromagnetic sources is continuously moved along the skin surface to apply electromagnetic energy to the tissue in the treatment area. In many embodiments, a continuous wave (CW) source of electromagnetic energy can be utilized in the scanning mode. In other embodiments, a pulsed source of electromagnetic energy can be employed in the scanning mode, as described in more detail below.

FIG. 7C schematically illustrates a “matrix” mode of applying electromagnetic radiation to a treatment area. More particularly, a device 76 according to one embodiment of the invention includes a composite EMR source 77 formed as an array of individually addressable EMR sources, such as LEDs, which can be activated simultaneously, sequentially, or in a selected pattern. A treatment area, e.g., a large treatment area such as a whole face, can be positioned near or in contact with a panel 75 of the device 76 to receive radiation from the array of the EMR sources. Further, a beam shaping and/or cooling implement 78 can be optionally employed to optimize the delivery of the electromagnetic radiation to the treatment area.

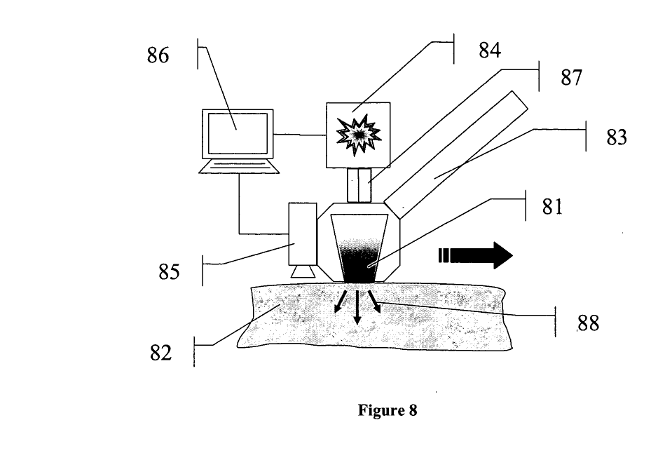

In some embodiments, it may be advantageous to utilize a pulsed source of EMR in the scanning mode. As shown schematically in FIG. 8, such an embodiment can employ a system that includes a pulsed source 84, a tracking device 85, and a triggering device 86, e.g., a computer. The system further includes a handpiece (applicator) 81 that delivers electromagnetic radiation to a treatment area 82. The pulsed source may or may not be integrated with the handpiece. In the latter case, radiation is delivered to the handpiece through an additional energy guide 87. The applicator can be equipped with a handle 83 for manual scanning. Alternatively, mechanical scanning can be used. A treatment session can be initiated by placing the applicator on the skin surface and firing the first pulse 88. Then the applicator is moved continuously along the skin surface, scanning the intended treatment area. The tracking device 85 continuously monitors position of the applicator and sends the data to the triggering device 86. Both devices may or may not be integrated with the handpiece and/or with each other.

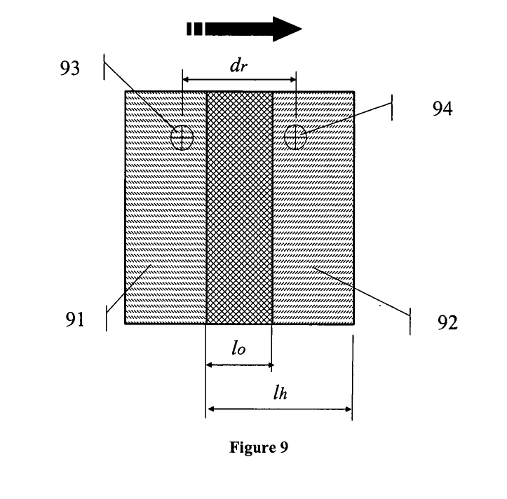

As shown schematically in FIG. 9, the triggering device compares the current position 92 of the handpiece with the last firing position 91 to initiate firing of a new pulse based on a predefined triggering condition. In one preferred embodiment, the triggering device selects a reference point (RP) on the applicator's frame, marks its position before the first pulse, and monitors the distance dr between the current (constantly changing due to scanning) position 94 of RP and its last firing position 93. For example, the triggering device can monitor the following condition:

dᵣ≧lₕ−lₒ=lₕ(1−α), (1)

where α=lₒ/lₕ is the desired degree of overlap, lₒ is the length of overlap [mm], and lₕ is the length of the handpiece's working area [mm]. Once the condition of Eq.(1) is fulfilled, the triggering device can issue a new firing command to the pulsed source. The procedure is repeated until the whole treatment area is covered. The firing command can be in the form of an analogue or digital pulse (or sequence of pulses) and can be transmitted to the pulsed source by a variety of mechanisms (for example, electrical, mechanical, or optical). Other triggering algorithms can be devised by those having ordinary skill in the art without departing from the scope of the present invention.

The tracking device 85 can be implemented by utilizing various techniques. For example, in one embodiment, the tracking device can include a set of wheels and a reading module, which reads angular positions of the wheels. Once the number of rotations corresponding to the Eq.(1) is made, a firing command is issued. In some preferred embodiments, in which the tracking device is a non-contact optical device, the skin surface can be illuminated, and a picture of a limited area can be taken with sufficient frequency (for example, 2 kHz). The sequence of pictures can then be processed, and the differences between the frames can be analyzed in such a way as to determine the shift between the camera positions at the instances when the frames were taken. As a result, position of RP can be reliably monitored. In some preferred embodiments, the tracking device can be a commercially available optical mouse (possibly modified to accommodate for the specific configuration of the application). The tracking device can also perform function of the contact sensor.

The triggering device 86 can be mechanical, electromechanical, electrical, electronic, optical or of any other suitable design. It can be either analogue or digital. In some preferred embodiments, the triggering device is an electronic digital device.

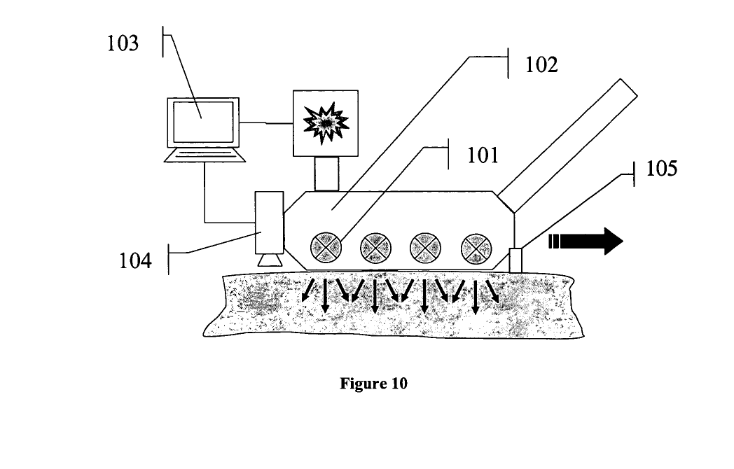

Some embodiments can employ composite EMR sources for delivering radiation to a selected skin treatment area, for example, via a stamping or a scanning mode. By way of example, with reference to FIG. 10, a plurality of EMR sources 101 can be organized in a linear array in a handpiece 102. When handpiece 102 is scanned along the skin surface, a timing device 103 can send firing pulses to the sources 101 according to a programmed sequence to activate all or selected ones of the EMR sources. In this manner, a desired effect on the target can be achieved due to cumulative action of the multiple pulses from the composite source. The firing sequence can also be modified “on the fly” as a function of, for example, scanning velocity or skin conditions (such as pigmentation or erythema), monitored by a tracking device 104. In some embodiments, the apparatus can also have a positioning mechanism 105 for positioning the hair for treatment. The positioning mechanism can be a mechanical, electrostatic, and/or vacuum source capable of moving a portion of the hair so that the hair can optimally receive the applied radiation.

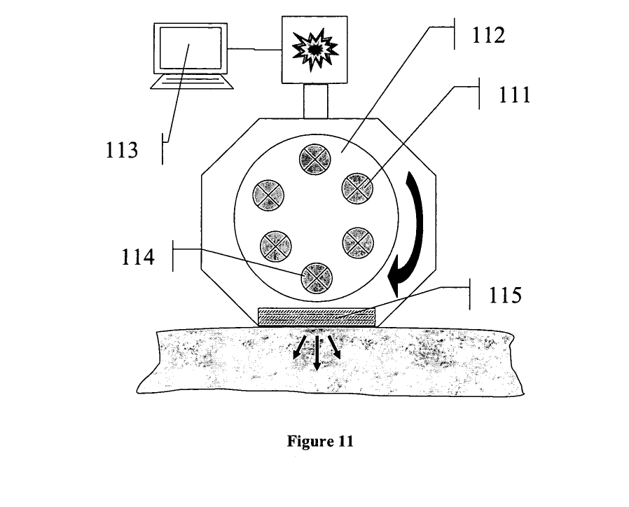

Alternatively, as shown in FIG. 11, the EMR sources 111 can be positioned in a rotating drum 112. The timing device 113 can generate a firing sequence in such a way as to fire each source in the time instant when the source occupies the “bottom” position 114, facing a portion of the treatment area. Beam-shaping and/or cooling implement 115 can be integrated within the handpiece housing. Other arrangements of sources in an array can be devised by those skilled in the art.

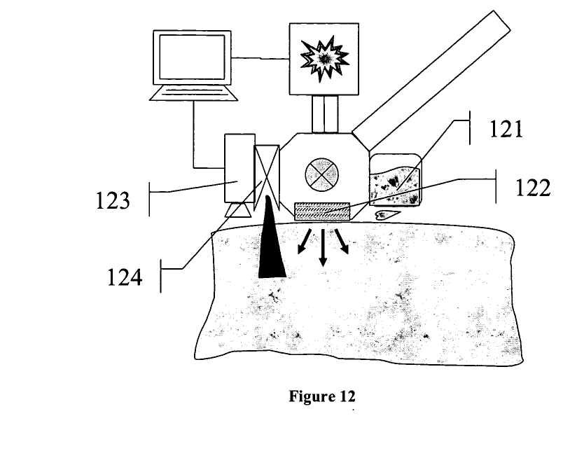

Some embodiments of an apparatus according to the teachings of the invention can include additional implements to further increase efficacy and/or safety of the treatment. For example, with reference to FIG. 12, the apparatus can include a topical-composition-dispensing implement 121, cooling implement 122, feedback (skin condition monitor) implement 123, or hair straightening implement 124. In addition, the apparatus can include implements, such as, razors, for depilating the treatment area.

In some embodiments, a single apparatus can be utilized for performing two or more treatment methods according to the teachings of the invention.

Example that follows provides further understanding of some aspects of the hair treatment methods according to the teachings of the invention.

### EXAMPLE 1

Comparison of the efficacy of 1064 nm wavelength vs 1208 nm wavelength for heating melanin-containing target (hair)

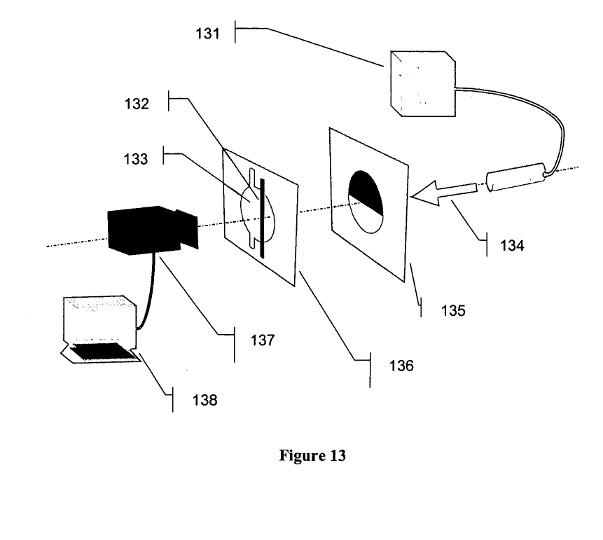

An experimental set-up shown schematically in FIG. 13 was employed to compare the efficiency of utilizing a 1208 nm radiation source relative to a 1060 nm source for heating low-melanin-content (“white”) and high-melanin-content (“black”) hairs.

A CW Raman fiber laser 131 was employed to generate radiation at 1060 nm and 1208 nm wavelengths. A 2-mm aperture 136 was utilized to select a portion of a radiation beam 134 having a maximum intensity (“flat top”). A black hair 132 and a white hair 133, which were harvested immediately before performing measurements to avoid de-hydration, were mounted in the beam path as symmetrically as possible relative to the beam's central point. Total incident power was matched for the two wavelengths at 240 mW (i.e., 7.6 W/cm² irradiance). An electronic shutter 135, controlled by a pulse generator, was utilized to generate ˜200-ms pulses at both wavelengths (resulting in ˜1.5 J/cm² fluence). An infrared thermal camera 137, controlled by a computer 138, was focused at the plane containing the hairs, and points of maximal temperature rise were selected at both hairs. Temporal profiles of the temperature at these points were recorded.

Results:

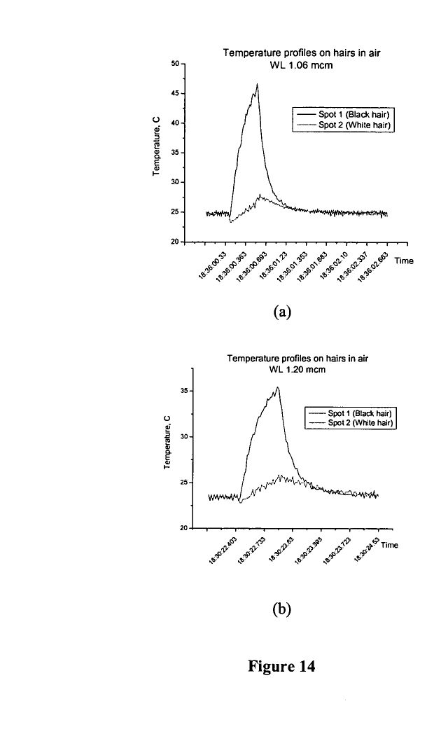

FIGS. 14A and 14B present, respectively, two temperature histories for each wavelength. Further, Table 1 below summarizes averaged temperature data for both hairs at the two wavelengths.

TABLE 1

| Wavelength, | Maximal temperature rise, ° C. | | Contrast |
|---|---|---|---|
| nm | White hair | Black hair | Black/White |
| 1060 | 4.8 | 21.8 | 4.5 |
| 1208 | 3.3 | 11.8 | 3.6 |
| Ratio | 1.5 | 1.8 | 1.25 |
| 1060/1208 | | | |

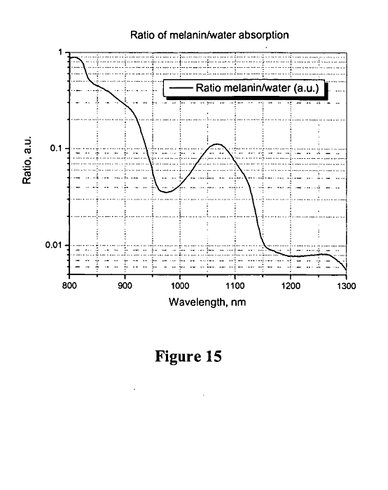

The contrast in temperature rise between the black and the white hairs does not change significantly between the two wavelengths, thus suggesting that melanin remains the dominant absorber at 1208 nm. If it were not so, the change in contrast would be closer to the change in the ratio of melanin/water absorption, shown in FIG. 15.

The ratio of the temperature rise at 1060 nm to that at 1208 nm is consistent with the melanin absorption spectrum in the IR (See FIG. 4). The data further indicates that absorption of melanin at 1208 nm appears to be still sufficient to induce substantial efficiency of heating (˜8 deg C/(J/cm²) in the present set-up).

Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims. All publications and references are herein expressly incorporated by reference in their entirety.

1. An apparatus for modifying a shape of a least a portion of a hair tip, comprising
at least a radiation source generating radiation pulses having wavelengths in a range of about 280 nm to about 100,000 nm and pulse widths in a range of about 1 nsec to about 5 minutes to illuminate a skin treatment area with a fluence in a range of about 0.01 J/cm² to about 1000 J/cm² so as to modify shapes of at least some hair tips in the treatment area.

2. The apparatus of claim 1, further comprising a mechanism for removing portions of the hair tips protruding above the skin surface.

3. An apparatus for reducing curliness of hair shafts, comprising
one or more radiation sources generating radiation pulses having wavelengths in a range of about 380 nm to about 2700 nm and pulse widths in a range of about 1 nsec to about 1 minute for illuminating a skin treatment area with a fluence in a range of about 0.1 J/cm² to about 1000 J/cm² so as to reduce curliness of at least some hair shafts in the treatment area.

4. The apparatus of claim 3, further comprising a mechanism for removing portions of the hair tips protruding above the skin surface.

5. An apparatus for controlling hair growth, comprising
at least one radiation source generating electromagnetic radiation having wavelength components in a range of about 1200 to about 1400 nm for application to one or more hair follicles in a skin treatment area so as to modulate hair growth,
wherein said radiation source can be any of an LED, a laser diode, a filtered arc lamp or a filtered halogen lamp.

6. An apparatus for modifying elasticity of hair shafts, comprising
one or more radiation sources generating radiation pulses having wavelengths in a range of about 600 to about 1400 nm and pulse widths in a range of about 1 nsec to about 1 minute for illuminating a skin treatment area with a fluence in a range of about 0.1 J/cm² to about 1000 J/cm² so as to modify elasticity of at least some hair shafts in the treatment area.

7. A dermatological system, comprising
an applicator having a head portion adapted for scanning over a skin treatment area and incorporating at least one radiation source,
a tracker coupled to said head portion for generating signals indicative of positions of said head portion during a scan, and
a controller coupled to said tracker and said radiation source, said controller periodically activating said radiation source based on position signals received from the tracker.

8. The apparatus of claim 7, wherein said controller determines a distance traversed by said head portion since a previous activation of said radiation source based on said position signals.

9. The apparatus of claim 8, wherein said controller activates the source when said traversed distance exceeds a threshold.

10. A hair treatment method comprising:
applying electromagnetic radiation (EMR) to a skin treatment area to deposit energy in one or more hair tips in the area so as to modify a shape of at least a portion of said hair tips.

11. The method of claim 10, wherein said step of applying radiation comprises exposing at least a portion of said treatment area to a plurality of EMR pulses.

12. The method of claim 10, wherein said applied radiation causes heating of said hair tips so as to reduce sharpness of said tips.

13. The method of claim 10, wherein said applied radiation modifies the shape of said hair tips to a substantially rounded shape.

14. The method of claim 10, wherein said applied radiation modifies the shape of said hair tips so as to inhibit extrafollicular and/or transfollicular penetration of said hair tips.

15. The method of claim 10, wherein said applied radiation causes any of treatment and/or prevention of pseudofolliculitis barbae (PFB) in the treatment area.

16. The method of claim 10, wherein said applied radiation raises temperature of said hair tips to a range of about 50 to about 300° C.

17. The method of claim 10, further comprising selecting said applied radiation so as to raise temperature of said hair tips to a range of about 50 to about 300° C. while keeping epidermal temperature in the treatment area below about 65° C.

18. The method of claim 11, wherein said pulses have pulse widths in a range of about 1 ns to about 5 minute.

19. The method of claim 11, wherein said pulses have pulse widths between about 1 microsecond to about 100 milliseconds.

20. The method of claim 19, wherein said pulses have a repetition rate ranging from about 0.1 Hz to about 1 MHz.

21. The method of claim 10, wherein said radiation applies a fluence in a range of about 0.01 J/cm² to about 1000 J/cm² to said treatment area.

22. The method of claim 10, wherein said applied radiation includes wavelength components in a range of about 280 nm to about 100000 nm.

23. The method of claim 10, wherein said applied radiation includes wavelength components in a range of about 380 nm to about 600 nm.

24. The method of claim 10, wherein said applied radiation includes wavelength components absorbed by at least one of melanin, water, and keratin in said hair tips.

25. The method of claim 10, further comprising drying hair tips in the treatment area prior to said application of the electromagnetic radiation.

26. The method of claim 25, further comprising delivering a flow of air over said treatment area to dry said hair tips.

27. The method of claim 10, further comprising the step of cooling the epidermis in the treatment area.

28. The method of claim 27, wherein said cooling step is performed at any of prior, during or after application of said radiation to the treatment area.

29. The method of claim 10, further comprising applying a topical agent to said skin treatment area, said topical agent being photoactivated chemically or thermally by said radiation to facilitate modifying the shape of the hair tips.

30. The method of claim 29, wherein said topical agent comprises at least one chromophore.

31. The method of claim 30, wherein said topical agent comprises a vehicle for delivering said chromophore to the pilosebaceous canal of hairs in said treatment area.

32. The method of claim 10, wherein said hair tips extend from about 0.2 mm below the skin surface to about 1 mm above the skin surface.

33. The method of claim 10, further comprises removing portions of hair tips protruding above the skin surface prior to applying said radiation.

34. The method of claim 33, wherein said step of removing portions of hair tips is performed substantially simultaneously with applying said electromagnetic radiation.

35. The method of claim 33, wherein the step of removing portions of the hair tips is selected from the group consisting of shaving, clipping, applying a depilatory cream, or applying additional electromagnetic radiation.

36. The method of claim 10, wherein the method further comprises stretching the skin treatment area.

37. The method of claim 10, wherein the method further comprises lifting hairs in the skin treatment area.

38. A method of treating hair, comprising
applying electromagnetic radiation to a skin treatment area for one or more hair shafts in the treatment area so as to cause a change in elasticity of said hair shafts.

39. The method of claim 38, wherein said radiation increases elasticity of said irradiated hair shafts.

40. The method of claim 38, wherein said radiation causes a change in a tensile strength of said hair shafts in a range of about 1 to about 200 MPa of breaking stress.

41. The method of claim 38, wherein said radiation causes substantial straightening of said hair shafts.

42. The method of claim 38, wherein said elasticity change of said hair shafts facilitates any of prevention or treatment of pseudofolliculitis barbae (PFB) in the treatment area.

43. The method of claim 38, wherein said elevated temperature is in a range of about 50° C. to about 300° C.

44. The method of claim 38, wherein said step of applying electromagnetic radiation comprises applying a plurality of electromagnetic pulses to said treatment area.

45. The method of claim 44, wherein said radiation includes wavelength components in a range of about 380 nm to about 2700 nm.

46. The method of claim 44, wherein said radiation includes wavelength components in a range of about 600 nm to about 1400 nm.

47. The method of claim 44, wherein said pulses have pulse widths in a range of about 1 nsec to about 1 minute.

48. The method of claim 47, wherein said pulses provide a fluence in a range of about 0.1 J/cm² to about 1000 J/cm².

49. The method of claim 44, further comprising cooling the epidermis in said treatment area.

50. The method of claim 44, further comprising applying a topical agent to said treatment area, said topical agent being capable of photoactivation by said radiation to facilitate softening of the hair shafts.

51. A method of controlling hair growth, comprising
applying electromagnetic radiation having wavelength components in a range of about 1200 to about 1400 nm to one or more hair follicles in a skin treatment area so as to modulate hair growth.

52. The method of claim 51, wherein said applied radiation causes a deceleration of hair growth.

53. The method of claim 51, wherein said applied radiation causes a cessation of hair growth.

54. The method of claim 51, wherein said applied radiation causes a stimulation of hair growth.

55. The method of claim 51, wherein said modulation of hair growth causes any of prevention or treatment of pseudofolliculitis barbae (PFB) in the treatment area.

56. The method of claim 51, further comprising selecting a fluence of said applied radiation to be in a range of about 0.1 J/cm² to about 1000 J/cm².

57. The method of claim 51, wherein the step of applying radiation comprises exposing the skin treatment area to a plurality of radiation pulses having pulse widths in a range of about 1 ns to about 1000s.

58. The method of claim 51, further comprising the step of cooling the epidermis in the treatment area.

59. The method of claim 51, further comprising selecting duration and fluence of said applied radiation so as to cause heating of at least a portion of said hair follicles.

60. A method of treating hair, comprising
irradiating a plurality of hair follicles in a treatment area with radiation of a wavelength, and fluence suitable for decreasing curliness of at least a portion of said hairs.

61. The method of claim 60, wherein said irradiated portion of the hair follicles comprises at least one of the hair bulb, keratogenous zone and bulbar of the hair follicles.

62. The method of claim 60, wherein said radiation causes the hair matrix to effect growth of thinner hair.

63. The method of claim 60, wherein said hair having reduced curliness exhibits a change in a tensile strength in a range of about 1 to about 200 MPa of breaking stress relative to that of a pre-treatment hair.

64. The method of claim 60, wherein said hair having reduced curliness exhibits a reduction in diameter in a range of about 1 to about 60 micrometers relative to that of a pre-treatment hair.

65. The method of claim 60, further comprising selecting said wavelength to be in a range of about 380 nm to about 2700 nm.

66. The method of claim 60, further comprising selecting said wavelength to be in a range of about 600 nm to about 1400 nm.

67. The method of claim 60, further comprising selecting said fluence to be in a range of about 0.1 J/cm² to about 1000 J/cm².

68. The method of claim 60, wherein said irradiating step comprises applying a plurality of electromagnetic pulses to said treatment area.

69. The method of claim 60, wherein said pulses have pulse widths in a range of about 1 ns to about 10 minute.
